(19) United States
(12) Patent Application Publication
(10) Pub. No.: US 2014/0209491 A1
(43) Pub. Date: Jul. 31, 2014

(54) Combination Beverage Container and Condom Holder

(71) Applicant: George Hillhouse, Austin, TX (US)

(72) Inventor: George Hillhouse, Austin, TX (US)

(21) Appl. No.: 14/166,353

(22) Filed: Jan. 28, 2014

Related U.S. Application Data
(60) Provisional application No. 61/849,558, filed on Jan. 29, 2013.

(51) Int. Cl. A61F 6/00

(52) U.S. Cl. CPC A61F 6/005
USPC 206/223

(57) ABSTRACT

A novelty beverage container such as a mug or a glass that is coupled to a condom is disclosed herein. The condom may be coupled to the mug in a variety of ways and in at least one embodiment the condom is contained in a compartment that is attached to or integral with the beverage container.

### CROSS REFERENCE TO RELATED APPLICATION

This patent application claims priority to provisional patent application No. 61/849,558 filed on Jan. 29, 2013 and entitled “Combination beverage container and Condom Holder”.

### TECHNICAL FIELD

The subject disclosure concerns a novelty beverage container such as a mug or a glass that is coupled to a condom.

### BACKGROUND OF THE INVENTION

The novelty product market is intended to make people laugh. There has never been a mug or wine glass combined with a condom. To certain people this combination may be funny. In certain cases it might actually be helpful as a last resort family planning tool.

What is needed is a novelty beverage holder that makes people laugh and in some circumstances may be a family planning tool.

Characteristics and advantages of the present disclosure described above and additional features and benefits will be readily apparent to those skilled in the art upon consideration of the following detailed description of various embodiments and referring to the accompanying drawings.

### BRIEF DESCRIPTION OF THE FIGURES

FIG. 1 shows a cross section of a mug with a compartment holding a condom.

FIG. 2 shows a cross section of a wine glass with a compartment holding a condom.

FIG. 3 shows a label with directions on how to use the novelty products of FIGS. 1 and 2.

### DETAILED DESCRIPTION OF THE INVENTION

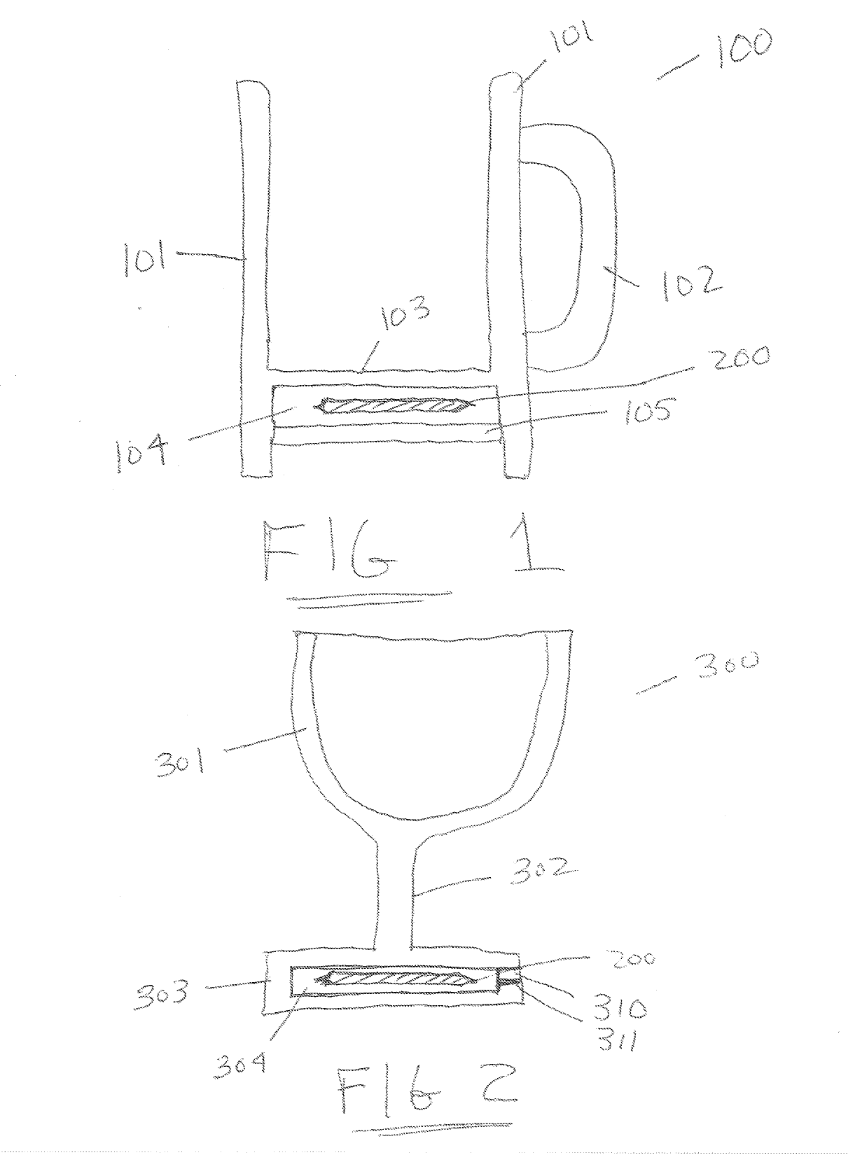

FIG. 1 shows a cross sectional drawing of mug 100. Mug 100 comprises handle 102, side wall 101, and base 103. Below and proximate to base 103 is compartment 104 that, as shown in FIG. 1, Is defined by base 103, wall 101 and sub base 105. Compartment 104 may be attached to mug 100 or an integral part of mug 100. Inside of compartment 104 is condom 200 (either loosely or securely in place).

As just of many examples of how condom 200 is placed into compartment 104, it may be possible to placed condom 200 into the area below base 103 and then in a second step sub base 105 can be glued into place, snapped into place, sonic welded into place and so forth. In some instances sub-base 104 can be an integral part of mug 100 and is formed when mug 100 is injection molded (if mug 100 is plastic and not glass).

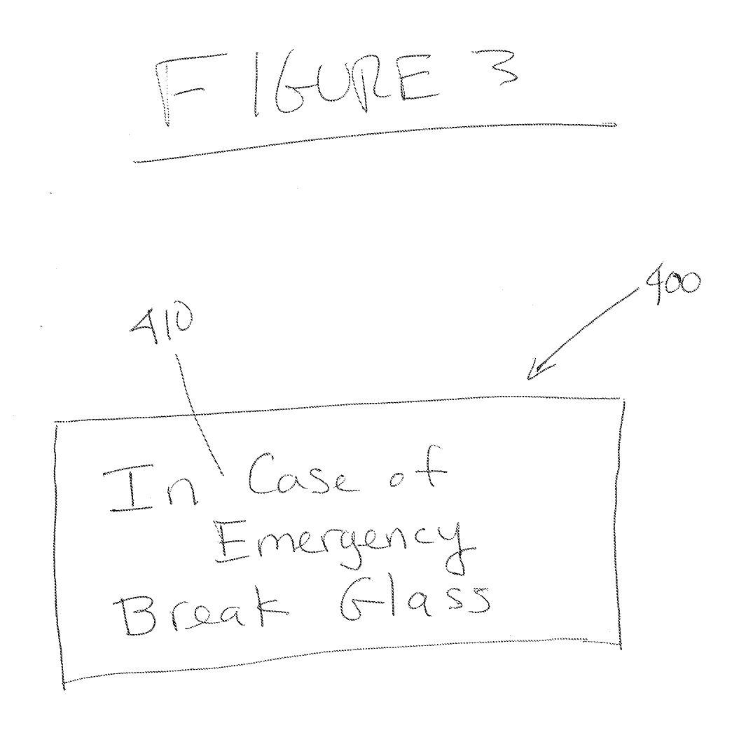

The placement of compartment 104 and condom 200 is unimportant. In fact, in some embodiments it may be desirable to put condom 200 in a more prominent position higher up on mug 100 (e.g. inside of side wall 101 or proximate to side wall 101). Placement of condom 200 in a higher position on mug 100 (or wine glass 300, see FIG. 2 supra) allows for the novelty aspect of mug 100 to be more obvious to the casual observer. On the other hand, some users may prefer the more subtle placement of the condom on the bottom of mug 100 as shown in FIG. 1.

FIG. 2 shows a cross section of wine glass 300 that comprises wall 301, stem 302 and base 303. Base 303 has compartment 304 that is sized and shaped to hold condom 200 (either loosely or securely). In the case of wine glass 300, compartment 304 may be formed as an integral part of wine glass 300 and condom 200 is later inserted into compartment 304 through opening 310 that is later plugged with plug 311. Plug 311 can be put into place by means well known in the prior art such as gluing, snapping, sonic welding and so forth. (A similar plugging of compartment 104 in mug 100 is yet another example of how condom 200 could be placed inside mug 100).

To increase the humor of this novelty product (or increase its functionality when used as a last resort family planning tool), it may be desirable to affix to the beverage container directions telling a user how they can access condom 200. FIG. 3 shows label 400 with directional phrase 410. An example of directional phrase 410 comprises the words:

### “In Case of Emergency Break Glass”

In some embodiments it may be desirable to affix directional phrase 410 directly onto the beverage container by printing on the beverage container rather than using label 400.

The various ways of incorporating the condom on the beverage holder are unimportant. In fact, in some instances the condom may be attached to the beverage holder without using a compartment and instead it is attached to the beverage holder using methods such as shrink wrap, Velcro, tape, hang tags, adhesives and so forth.

Characteristics and advantages of the present disclosure and additional features and benefits will be readily apparent to those skilled in the art upon consideration of the following detailed description of exemplary embodiments of the present disclosure and referring to the accompanying figures. It should be understood that the description herein and appended drawings, being of example embodiments, are not intended to limit the claims of this patent or any patent or patent application claiming priority hereto.

On the contrary, the intention is to cover all modifications, equivalents and alternatives falling within the spirit and scope of the claims. Many changes may be made to the particular embodiments and details disclosed herein without departing from such spirit and scope.

In showing and describing preferred embodiments in the appended figures, common or similar elements are referenced with like or identical reference numerals or are apparent from the figures and/or the description herein. The figures are not necessarily to scale and certain features and certain views of the figures may be shown exaggerated in scale or in schematic in the interest of clarity and conciseness. As may be used herein and throughout various portions (and headings) of this patent application, the terms “invention”, “present invention” and variations thereof are not intended to mean every possible embodiment encompassed by this disclosure or any particular claim(s).

Thus, the subject matter of each such reference should not be considered as necessary for, or part of, every embodiment hereof or of any particular claim(s) merely because of such reference. The terms “coupled”, “connected”, “engaged” and the like, and variations thereof, as used herein and in the appended claims are intended to mean either an indirect or direct connection or engagement. Thus, if a first device couples to a second device, that connection may be through a direct connection, or through an indirect connection via other devices and connections.

Certain terms are used herein and in the appended claims to refer to particular components. As one skilled in the art will appreciate, different persons may refer to a component by different names. This document does not intend to distinguish between components that differ in name but not function. Also, the terms “including” and “comprising” are used herein and in the appended claims in an open-ended fashion, and thus should be interpreted to mean “including, but not limited to . . . .” Further, reference herein and in the appended claims to components and aspects in a singular tense does not necessarily limit the present disclosure or appended claims to only one such component or aspect, but should be interpreted generally to mean one or more, as may be suitable and desirable in each particular instance.

What is claimed is:

1. A novelty product comprising a beverage container coupled to a condom.

2. The novelty product of claim 1 wherein the condom is in a compartment that is attached to or integral with the container.

3. The novelty product of claim 2, wherein the beverage container is a mug with a base and a side wall.

4. The novelty product of claim 1 further comprising a directional phrase affixed to the beverage container.

5. The novelty product of claim 4 wherein the directional phrase comprises “In case of emergency break glass”.

5. The novelty product of claim 3 wherein the condom is proximate to the base of the mug.

6. The novelty product of claim 3 wherein the condom is proximate to the side wall of the mug.

7. The novelty product of claim 1 wherein the condom is coupled to the beverage container using shrink-wrap, tape, Velcro, snaps, hang tags or adhesives.

8. The novelty product of claim 2, wherein the beverage container is a glass with a base and a side wall.

9. The novelty product of claim 8 wherein the condom is proximate to the base of the glass.

10. The novelty product of claim 8 wherein the condom is proximate to the side wall of the glass.
